(19) United States
(12) Patent Application Publication
(10) Pub. No.: US 2006/0235325 A1
(43) Pub. Date: Oct. 19, 2006

(54) Congestive heart failure monitor

(76) Inventors: Nils Holmstrom, Jarfalla (SE); Martin Obel, Danderyd (SE); Anna Norlin, Stockholm (SE); Kenneth Dahlberg, Stockholm (SE); Anders Bjorling, Jarfalla (SE); Sven Kalling, Taby (SE); Karin Ljungstrom, Hasselby (SE); Kjell Noren, Solna (SE)

Correspondence Address: SCHIFF HARDIN, LLP;PATENT DEPARTMENT, 6600 SEARS TOWER, CHICAGO, IL, 60606-6473, US

(21) Appl. No.: 10/524,196

(22) PCT Filed: Jun. 23, 2003
(86) PCT No.: PCT/SE03/01081

§ 371 Date: Feb. 23, 2006

(30) Foreign Application Priority Data
Jul. 22, 2002 (SE) 0202289-5

(51) Int. Cl. A61B 5/05; A61B 5/02

(52) U.S. Cl. 600/547; 600/508

(57) ABSTRACT

A congestive heart failure monitor has an impedance measuring unit that measures the impedance between at least two electrodes implanted in a patient, to use a detected change of the measured impedance as an indication of a change in the left atrium volume. Any analyzing unit analyzes the measured impedance and detects insipient congestive heart failure dependent on a quotient of a maximum value of the measured impedance and a minimum value of the measured impedance during a cardiac cycle.

### BACKGROUND OF THE INVENTION

1. Field of the Invention

The present invention relates to a congestive heart failure monitor.

2. Description of the Prior Art

Electrical stimulation therapy of congestive heart failure is known. Thus in U.S. Pat. No. 5,584,868 a dual-chamber pacemaker designed for treating congestive heart failure (CHF) by changing the AV interval is described and in U.S. Pat. No. 6,223,079 a four chamber pacing system for improving cardiac output of CHF patients by controlling pacing to maintain the ventricular mechanical synchronization is disclosed. For providing suitable timing in the latter system impedance sensing in the left heart is used.

Incipient CHF is often present without the patient knowing it. An indicator for incipient CHF would therefore be of great value since treatment by addition of drugs or electrical stimulation therapy could then be introduced at an early stage of CHF to slow down the progression of CHF. This would prolong the survival of the patient. Such an indicator could also be used to alert the patient or the physician about new conditions so appropriate measures can be taken. The first sign of a CHF can be seen in the left atrium, for instance in volume changes thereof.

### SUMMARY OF THE INVENTION

An object of the present invention is to utilize the above-described knowledge to provide a congestive heart failure monitor for detecting CHF at an early stage.

The first sign of a CHF can be observed in the left atrium of the heart by monitoring its mechanical behavior, like volume changes, as mentioned above. If the pumping ability of the left ventricle is reduced the volume of the left atrium will increase due to the excessive filling of blood. The filing pattern of the left atrium can be disturbed due to mitral regurgitation caused by either diastolic or systolic dysfunction. The diastolic dysfunction could be a result of prolonged PR interval, i.e. the P-wave to QRS interval, or too long an AV interval, resulting in reversed flow back to the left atrium during diastole because the mitral valve does not close immediately after the atrial contraction. The systolic dysfunction could be a result of infarctic areas in the left ventricle, which disturbs the left ventricle contraction propagation so that the mitral valve cannot close properly, (the papillar muscle becomes asynchronous), bringing reversed flow back to the left atrium during systole. The systolic dysfunction in the left ventricle could also be a result of bad timing of the right and left ventricle stimulations (e.g. septum, innervated at RVOT stimulation, is involved in the left ventricle contraction) causing mitral regurgitation and disturbed filling pattern of the left atrium. All these conditions result in a disturbed-filling pattern of the left atrium, which is one of the first signs of CHF.

A first sign of CHF can thus be observed in the left atrium and since the conductivity of blood is different from that of tissue the monitor according to the invention has an impedance measuring unit that measures impedance between at least two electrodes intended to be implanted in the patient such that a change in the left atrium volume results in a change in the measured impedance. In this way not only incipient CHF can be detected but also the monitor according to the invention can be used as a diagnostic tool for studying the progression or regression of CHF for enabling proper treatment of the patient.

In an embodiment of the monitor according to the invention the analyzing unit includes an averaging unit that forms a mean (average) value of the measured impedance during a number of cardiac cycles and the analyzing unit analyzes the mean value to detect CHF. Alternatively, the analyzing unit can include a quotient determining unit that determines the quotient between the impedance minimum and maximum values during a cardiac cycle, and the analyzing unit analyzes the quotient to detect CHF. Preferably the analyzing unit analyzes both the impedance mean value and the quotient to detect CHF. Firstly, even though the impedance changes continuously during the heartbeat, the mean value will decrease when the left atrium volume increases. Secondly, the quotient between the impedance minimum and maximum values will be larger, with increasing blood filling of the left atrium. Accordingly with the present invention an efficient CHF monitor is provided based on the analysis of these two quantities.

In a further embodiment of the monitor according to the invention the electrodes are designed for implantation in the right and left atria, respectively, or for implantation in the right atrium and left ventricle. In an implantable monitor, one of the electrodes can be designed for implantation in the left atrium and the other electrode be formed by the outer capsule of the monitor, e.g. the pacemaker capsule when the monitor is included in a pacemaker.

Also other combinations of the above mentioned electrodes can be used for the impedance determination.

The electrodes intended for implantation in the left atrium and the left ventricle are preferably designed for implantation in a coronary vein. For all these alternatives signals corresponding to the blood filling of the left atrium are obtained from the electrodes.

In another embodiment of the monitor according to the invention the impedance measuring unit includes a measuring circuit in the form of synchronous demodulator for obtaining both the real and imaginary parts of the impedance, and the impedance measuring unit preferably determines the impedance phase angle for detecting and the analyzing unit analyzes the phase angle for detecting an incipient CHF. Since blood is resistive, a high degree of blood filling results in a small phase angle. On the contrary, if more heart tissue is present, as in a healthy heart, the phase angle will exhibit a larger negative value.

### DESCRIPTION OF THE DRAWINGS

FIG. 1 schematically illustrates an impedance measurement performed in an embodiment of the monitor according to the invention.

FIG. 2 is a schematic illustration of the basic components of the monitor according to the invention.

FIG. 3-5 respectively illustrate alternatives for performing impedance measurements in the monitor according to the invention.

FIG. 7 is a flow chart showing the basic steps in one embodiment of the operation of the monitor according to the invention.

### DESCRIPTION OF THE PREFERRED EMBODIMENTS

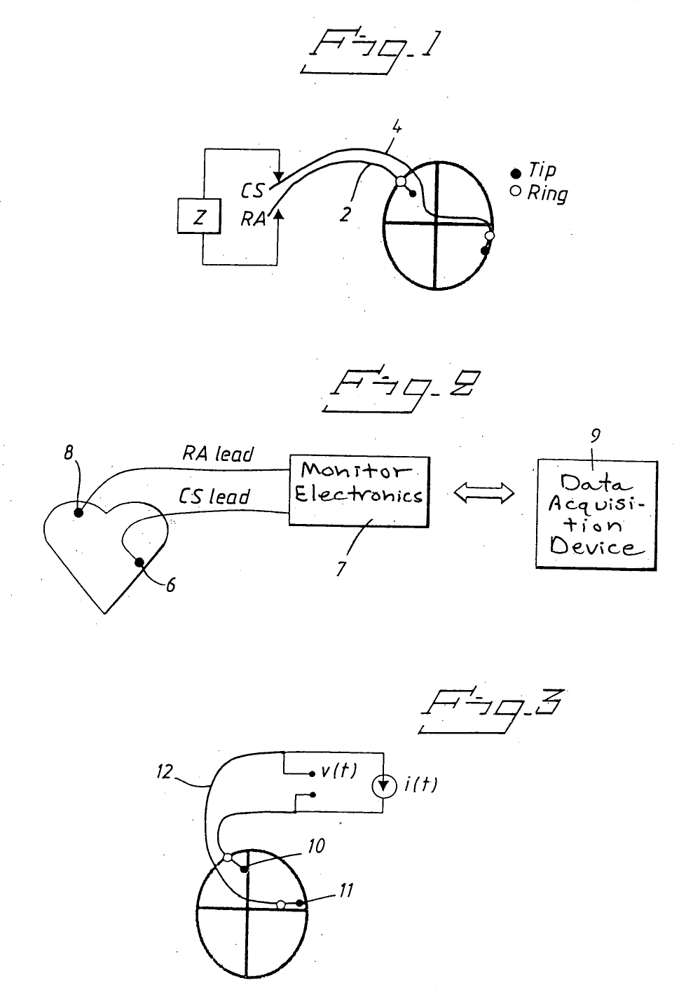

FIG. 1 illustrates measurement of the impedance Z between the right atrial lead 2 and the coronary sinus lead 4. As the left atrium is dilated due to CHF the impedance Z will decrease. Also the variation of the impedance between maximum and minimum values will then decrease due to increased wall tension.

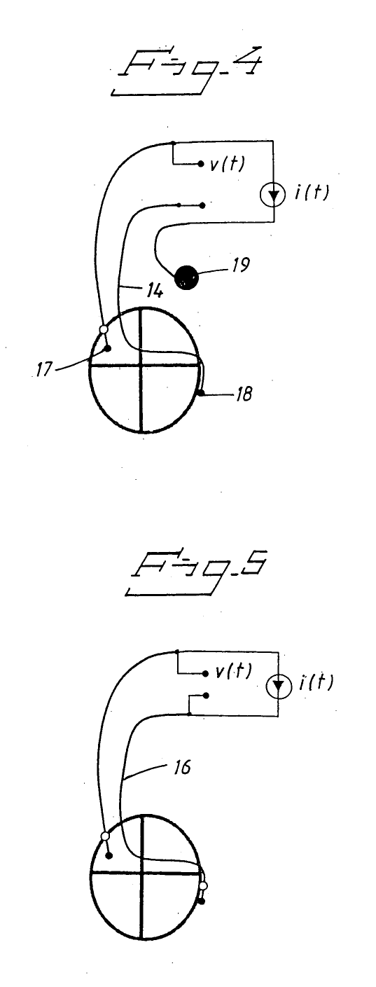

To secure a safe fixation of the left atrial electrode 6 in the coronary sinus CS or the great cardiac vein it is beneficial to use a screw-in electrode, cf. FIG. 2. The optimal right atrial RA electrode 2 position is lightly to be in the inter-atrial septum near the coronary sinus ostium, see the electrode tip 10 in FIG. 3. With the electrodes 6, 8; 10, 11 positioned as shown in FIGS. 2 and 3 the volume of the left atrium is positioned between the electrodes. This enables variations of impedance variations across the left atrium and a high sensitivity to left atrium volume changes.

Also, other bipolar electrode measurements set-ups as well as tripolar electrode settings are possible in the monitor according to the invention.

The embodiment of the monitor according to the invention shown in FIG. 2 includes monitor electronics 7 for analysis of the measured impedance for detection of an incipient CHF. An implantation monitor is preferably also provided with telemetry means, not shown in FIG. 2, for communication with an external programmer and data acquisition device 9.

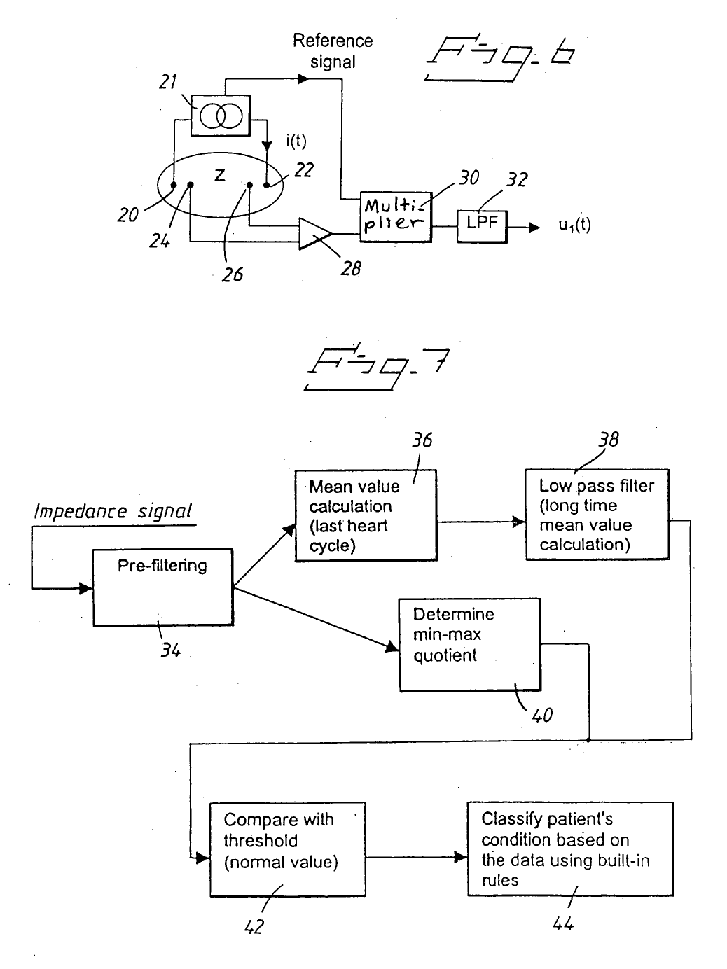

FIG. 3-5 illustrate quadropolar electrode configurations suitable for use in the monitor according to the invention. In FIG. 3 the coronary sinus CS lead 12 is positioned on the left atrium and in the FIGS. 4 and 5 the CS lead 14 and 16 respectively is placed on the left ventricle.

The method of bio-impedance measurement is not critical in the monitor according to the invention. FIG. 3-5 illustrate a technique wherein an electric current i(t) is supplied between two electrodes and the resulting evoked voltage response v(t) is detected. In the embodiments shown in FIGS. 3 and 5 the evoked voltage response i(t) is supplied. FIG. 4 shows an embodiment in which the current i(t) is supplied between a right atrial electrode 17 and a stimulator can 19, whereas the evoked voltage response is measured between the right atrial electrode 17 and a left ventricular electrode 18 positioned in the coronary sinus.

FIG. 6 shows an alternative embodiment of the impedance-measuring unit of the monitor according to the invention in the form of a synchronous demodulator. Generally the electric current i(t) is applied to two electrodes 20, 22 and the resulting evoked response is measured between two measurement electrodes 24 and 26. The measured voltage signal is amplified in an amplifier 28. The measured voltage signal is synchronized with the current i(t) with the aid of a reference signal picked up from the current source 21 and supplied to a synchronizing unit in the form of multiplier 30. A low-pass filter 32 is provided to filter the output signal from the multiplier 30. The resulting impedance Z₁ is the given by the expression
Z₁=u₁/i
where u₁ denotes the filtered resulting synchronized output voltage signal.

With the impedance measuring circuit shown in FIG. 6 both the real and the imaginary parts of the impedance are measured and consequently the impedance phase angle is obtained as well.

As discussed above, at left ventricular dysfunction the left atrium will dilate according to the progress of the disease, because the left ventricle is not able to eject blood into the body and blood will consequently stagnate in the left atrium and pulmonary veins. Left atrium blood pressure will increase as well as left atrium wall tension. The blood volume in the left atrium will also increase while the variation between maximum and minimum volume values will decrease. These phenomena can be determined from the measured impedance.

FIG. 7 is a flow chart illustrating an example of an embodiment of the monitor according to the invention analyzing the impedance minimum-maximum quotient and the overall impedance mean value for detecting an early CHF. The impedance raw signal obtained as explained above is pre-filtered, at 34 in FIG. 7. The filtering at 34 is performed to remove artifacts of noise, breathings etc. The mean (average) value of the impedance signal during the last heart cycle is calculated in averaging, at 36, and long time mean value calculation is performed by a low pass filter, at 38. The expression “long time” could mean a time of the order of typically 10 minutes in this connection.

At 40 in FIG. 7 the quotient between the impedance minimum and maximum values is determined. The obtained long term mean value and the quotient between minimum and maximum values are compared with predetermined reference or normal threshold values in comparison means, at 42 in FIG. 7. The results of these comparisons are used, at 44, to classify the patient's condition according to predetermined built-in rules.

The processing described above with reference to FIG. 7 can advantageously be used together with an activity sensor and a posture sensor. The impedance properties can then be calculated during the same conditions for the patient, for instance with the patient in a resting supine position. The processing chain of FIG. 7 can also preferably contain a memory for saving the time history of calculated parameters for further evaluation in external devices, cf. FIG. 2.

Although modifications and changes may be suggested by those skilled in the art, it is the invention of the inventors to embody within the patent warranted heron all changes and modifications as reasonably and properly come within the scope of their contribution to the art.

1-17. (canceled)

18. A congestive heart failure monitor comprising:
an impedance-measuring unit having two electrodes adapted to interact with a patient to measure an impedance representative of a change in a volume of the left atrium of the heart of the patient; and
an analyzing unit supplied with a signal representing said impedance, said analyzing unit including a quotient determining unit that determines a quotient between a minimum value of said impedance and a maximum value of said impedance during a cardiac cycle of said heart, said analysis unit detecting congestive heart failure dependent on said quotient.

19. A monitor as claimed in claim 18 wherein said analysis unit includes an averaging unit that forms an average value of said impedance during a plurality of cardiac cycles of said heart, and wherein said analysis unit additionally uses said average value to detect congestive heart failure.

20. A monitor as claimed in claim 19 comprising a comparator that compares said average value with a predetermined impedance threshold value, to obtain a comparison result, and wherein said analyzing unit detects congestive heart failure dependent on said comparison result.

21. A monitor as claimed in claim 20 wherein said averaging unit forms a floating average value of said impedance during a predetermined number of preceding cardiac cycles for use as said impedance threshold value.

22. A monitor as claimed in claim 18 wherein said analyzing unit comprises a quotient averaging unit that forms an average value of said quotient over a plurality of cardiac cycles of said heart, and wherein said analyzing unit uses said average value of said quotient to detect congestive heart failure.

23. A monitor as claimed in claim 22 wherein said analysis unit comprises a comparator that compares said average value of said quotient with a predetermined quotient threshold value, to obtain a comparison result, and wherein said analyzing unit detects congestive heart failure dependent on said comparison result.

24. A monitor as claimed in claim 23 wherein said quotient averaging unit forms a floating average value of said quotient during a predetermined number of preceding cardiac cycles for use as said quotient threshold value.

25. A monitor as claimed in claim 18 comprising an averaging unit that forms an average value of said impedance during a plurality of cardiac cycles, and a comparator that compares said average value of said impedance to a predetermined impedance threshold value, and a quotient averaging unit that forms an average value of said quotient over said plurality of cardiac cycles, said comparator also comparing said average value of said quotient to a predetermined quotient threshold value to obtain a second comparison result, and said analysis unit detecting congestive heart failure dependent on both said first and second comparison results.

26. A monitor as claimed in claim 18 wherein said electrodes are adapted respectively for implantation in the right atrium and the left atrium of said heart.

27. A monitor as claimed in claim 18 wherein said electrodes are adapted for implantation respective in the right atrium and the left ventricle of said heart.

28. A monitor as claimed in claim 18 comprising a housing adapted for implantation in said patient, said housing containing said impedance-measuring unit and said analyzing unit, and wherein a first of said electrodes is adapted for implantation in the left atrium of said heart, and a second of said electrodes is formed by an exterior of said housing.

29. A monitor as claimed in claim 18 wherein said electrodes are adapted for implantation respective in the left atrium of said heart and the left ventricle of said heart, proceeding in a coronary vein.

30. A monitor as claimed in claim 18 wherein said impedance-measuring unit comprises a measuring circuit formed by a synchronous demodulator for obtaining both real and imaginary parts of said impedance.

31. A monitor as claimed in claim 18 wherein said impedance-measuring unit determines a phase angle of said impedance and wherein said analyzing unit analyzes said phase angle to detect congestive heart failure.

32. A multi-site heart stimulator comprising:
a stimulation unit adapted to interact with cardiac tissue to electrically stimulate said cardiac tissue with pacing pulses;
an impedance-measuring unit having two electrodes adapted to interact with a patient to measure an impedance representative of a change in a volume of the left atrium of the heart of the patient, and an analyzing unit supplied with a signal representing said impedance, said analyzing unit including a quotient determining unit that determines a quotient between a minimum value of said impedance and a maximum value of said impedance during a cardiac cycle of said heart, said analysis unit detecting congestive heart failure dependent on said quotient; and
a control unit connected to said stimulation unit and to said monitor for controlling delivery of said pacing pulses by said stimulation unit dependent on detection of congestive heart failure by said monitor.
